(19) United States
(12) Patent Application Publication
(10) Pub. No.: US 2011/0059510 A1
(43) Pub. Date: Mar. 10, 2011

(54) TRANSGENIC RABBITS PRODUCING HUMAN FACTOR VII

(76) Inventor: Pierre-Jean Ripoll, Longpont Sur Orge (FR)

(21) Appl. No.: 12/934,124

(22) PCT Filed: Mar. 25, 2008
(86) PCT No.: PCT/FR08/00396

§ 371 Date: Nov. 17, 2010

(51) Int. Cl. C12N 9/48; A01K 67/027; A23C 9/00

(52) U.S. Cl. 435/212; 800/14; 426/580

(57) ABSTRACT

The invention relates to transgenic rabbits that produce human factor VII in their mammary glands. The milk of said transgenic rabbits can be used as a raw material for the production of recombinant human factor VII.

The present invention relates to the production of recombinant human factor VII in the milk of transgenic rabbits.

Factor VII is a plasma protein involved in the blood coagulation process and in particular in initiating the extrinsic coagulation pathway. Factor VII, which is a vitamin K-dependent glycoprotein, is synthesized in the liver in the form of a precursor of 466 amino acids comprising a signal peptide (residues 1-20) and a propeptide (residues 21-60). Factor VII circulating in blood plasma is a zymogen (or proenzyme) constituted of a peptide chain of 53 kDa containing 406 residues. It results from several post-translational modifications: cleavage of the signal peptide and of the propeptide, gamma-carboxylation of the first ten N-terminal glutamic acids at positions 6, 7, 14, 16, 19, 20, 25, 26, 29 and 35, partial β-hydroxylation of the aspartic acid at position 63, O-glycosylation of the serines at positions 52 and 60, respectively with a glycose(xylose)₀₋₂ unit and with a fucose unit, and N-glycosylation of the asparagines at positions 145 and 322 with complex, predominantly biantennary and sialylated, structures. Factor VII is activated to give factor VIIa by proteolytic cleavage between the arginine at position 152 and the isoleucine at position 153: factor VIIa is composed of a light chain of 152 amino acids, derived from the N-terminal end, having a molecular mass of approximately 20 kDa and of a heavy chain of 254 amino acids, derived from the C-terminal end, having a molecular mass of approximately 30 kDa, which are covalently bonded to one another by a disulfide bridge between the cysteines at positions 135 and 262.

Circulating factor VII can complex with tissue factor (TF) produced by subendothelial fibroblasts, when the latter is released on the occasion of a breach in the vascular endothelium. The formation of this complex is accompanied by activation of factor VII to give factor VIIa. The TF-VIIa complex activates factors IX and X, leading to the formation of factors IXa and Xa, which in turn activate the conversion of prothrombin to thrombin, which allows the conversion of fibrinogen to fibrin, resulting in clot formation.

Factor VII/VIIa has been used for many years to treat patients suffering from various hemostasis disorders (by way of example, mention will be made of hemophilia type A which corresponds to a factor VIII deficiency, hemophilia type B which corresponds to a factor IX deficiency, or hereditary factor VII/VIIa deficiencies), and also hemorrhagic events of various origins, for instance strokes or hemorrhagic traumas.

Initially, the factor VII preparations used were obtained from human plasmas. However, owing to purification difficulties, health risks associated with use of blood-derived products, and the limitation of human plasma supplies, preparations of recombinant factor VII, produced by mammalian cells transformed so as to express human factor VII, as described, for example, in application EP 0 200 421, are currently preferred over said plasma-derived preparations.

An alternative to the production of proteins of therapeutic interest by transformed cell cultures is their production by transgenic animals, and in particular in the milk of said transgenic animals. This approach theoretically has many advantages, and in particular a higher yield, a lower production cost than production in cell cultures, and an easier and more flexible increase in production capacity at the industrial level. However, although the possibility of producing, in transgenic mouse milk, a recombinant protein, plasminogen inhibitor (tPA), having biological properties similar to those of the native human protein was demonstrated many years ago (Gordon et al., Bio Technol, 5, 1183-7, 1987), the number of proteins which could be produced successfully in the milk of transgenic animals under conditions compatible with the development and the industrial production of a medicament for human use still remains very limited. This is because many factors, specific, firstly, to the protein of interest to be expressed and, secondly, to the host species in which it is desired to express it, can condition success or failure for a given protein of interest/host species combination.

Among these factors are, in particular, the ability of the host species to produce, in an industrializable amount, proteins having post-translational modifications similar to those of the native human protein. This factor is particularly essential if it is envisioned to produce, in recombinant form in milk, proteins such as factor VII, the post-translational modifications of which are many and varied (cleavage of the signal peptide and of the propeptide, γ-carboxylation, β-hydroxylation, O-glycosylation and N-glycosylation) and which are naturally produced in the liver. This is because the cells of the mammary gland have post-translational modification capacities that are different than those of hepatic cells.

It has, for example, been observed that transgenic mice expressing human protein C in their mammary glands cannot correctly carry out the propeptide cleavage and the γ-carboxylation (Drohan et al., Transgenic Res., 355-64, 1994). Similar observations have been made in transgenic pigs, for protein C and for factor IX (Lee et al., J. Biochem., 118, 81-7, 1995; Van Cott et al., Genet Anal., 15, 155-60, 1999).

Furthermore, O-glycosylation and N-glycosylation of proteins can vary, qualitatively and quantitatively, depending on the mammalian species concerned. Thus, human proteins do not contain a galactosyl galactose α(1-3) galactose motif (Galα1-3Gal motif) owing to the fact that the gene encoding alpha-1,3-galactosyl-transferase, which is responsible for the synthesis of this motif, is inactivated in humans and Old World (Europe, Asia) monkeys, whereas it is present in the other mammals.

This motif (also called α-Gal epitope) is consequently very immunogenic in humans (Galili et al., Blood, 82(8): 2485-2493, 1993). A glycosylated human protein produced in recombinant form in mammals having an active alpha-1,3-galactosyltransferase is therefore capable of providing a large amount of Galα1-3Gal motifs and risks causing considerable undesirable immune reactions.

However, the inventors have now found that, surprisingly, transgenic rabbits expressing in their mammary glands a sequence encoding human factor VII produce, in their milk, a recombinant factor VII which is correctly cleaved and, in addition, does not contain or contains only very few Galα1-3Gal motifs.

Consequently, the subject of the present invention is a transgenic rabbit expressing a human factor VII in its milk.

A transgenic rabbit in accordance with the invention contains, in its genome, one or more copies of a transgene comprising a polynucleotide encoding a human factor VII, placed under the transcriptional control of a promoter enabling its specific expression in the cells of the mammary glands of said rabbit.

The term “transgene” is intended to mean a nucleic acid construct stably inserted into the genome of a host organism, which is transmitted to its progeny from generation to generation. In the present case, the transgene allows the expression of a protein of interest (factor VII) in the milk of the transgenic host animal.

Promoters allowing specific expression in the cells of the rabbit mammary gland are known in themselves. They may, for example, be promoters of genes for caseins or for milk serum proteins; mention will in particular be made of the α-, β- or κ-casein promoters, the β-lactoglobin promoter, the α-lactalbumin promoter, the WAP (whey acidic protein) promoter or the lactoferrin promoter. It may be a promoter originating from the rabbit, for instance the WAP promoter described in application EP0527063, or a promoter originating from another mammalian species.

The polynucleotide encoding human factor VII is preferably a cDNA, or the coding portion (ORF) thereof. It may be the natural cDNA of factor VII, the sequence of which is known in itself, and available on the databases, for example under accession number M13232, or a sequence encoding a variant of human factor VII modified in particular so as to increase its activity and/or to suppress its undesirable effects; by way of examples, mention may be made of the variants described in application 2008/0010693 or that described in the publication by Sorensen et al. (Br. J. Haematol., 137(2): 158-65, 2007).

Use may advantageously be made of a DNA sequence optimized for expression in rabbit mammary glands. Such a sequence can be obtained in silico by techniques well known to those skilled in the art, in order to eliminate the cryptic splice sites, the mRNA-destabilizing A/T rich sequences, the polyadenylation sites and the TATA boxes which are potentially parasitic, and the CpG islands, and to optimize the codons in order to reflect the preferences of rabbit mammary gland cells for producing milk proteins.

Advantageously, the transgene contains, in addition to the promoter and the sequence encoding factor VII, other elements intended to optimize the transcription and/or the translation of the recombinant protein. Such elements are known in themselves to those skilled in the art (cf., for example, Houdebine et al., in: Carl A. Pinkert (ed), Vector Design for Transgene Expression Transgenic Animal Technology, 2nd edn, New York: Academic Press., 419-458, 2002).

Said element may in particular be:
a strong insulator, placed 5′ of the promoter, guaranteeing a level of expression of the protein dependent on the number of copies of transgene integrated and independent of the site of integration of the transgene in the genome of the animal: mention will, for example, be made of the 5′HS4 region of the chicken beta-globin gene (Taboit-Dameron et al., Transgenic. Res., 8: 223-235, 1999; Rival-Gervier et al., Transgenic. Res., 12: 723-730, 2003);
one or more exon/intron pairs that may contain one or more transcription or translation enhancers: by way of examples, mention may be made of: the introns of the early and late genes of the SV40 virus genome, the first intron of a beta-globin gene, the introns of the EF1-alpha gene, the introns of the alpha-s1 casein gene, the introns of the WAP gene, the introns of the human and bovine growth hormone genes; the enhancer sequences can in particular be chosen from those present in the LTR sequences of the HTLV virus, or the MMTV virus (murine mammary tumor virus), the enhancer sequence of the immunoglobulin gene, the enhancer sequence of the alpha-s1 casein gene, and the enhancer sequence of beta-globin. The enhancer sequence may also be the distal region upstream (up to 140 kbp) of the WAP gene or the distal region downstream (at least 10 kbp) of the WAP gene, as described by Rival-Gervier et al. (Mol. Reprod. Develop., 63: 161-167, 2002), in application EP 1 217 071;
a strong terminator guaranteeing efficient termination of transcription: by way of examples, mention will be made of the terminators of the SV40 virus early or late genes, and those of the beta-globin genes, of the WAP genes, or of the human or bovine growth hormone genes.

The transgenesis can be carried out by the conventional methods known in themselves. Advantageously, the transgene is introduced by microinjection into the pronuclei of fertilized embryos which are then reimplanted in carrier females. It is also possible to obtain the transgenic animals by nuclear transfer cloning followed by embryo transfer into recipient females.

The subject of the present invention is also the milk produced by the transgenic rabbits in accordance with the invention, and the use of this milk as a starting material for the purification of recombinant human factor VII.

The recombinant human factor VII can be purified from this milk by methods known in themselves, for example as described in U.S. Pat. No. 6,268,487.

The present invention will be understood more clearly from the further description which follows, which refers to nonlimiting examples describing the production of transgenic rabbits expressing factor VII in their milk.

### EXAMPLE 1

### Construction of a Transgenesis Vector Containing the Sequence Encoding Human Factor VII

Cloning of the Factor VII cDNA by PCR in an Intermediate Vector

A DNA sequence encoding human factor VII, bordered by the unique restriction sites MluI in the 5′ position and NheI in the 3′ position, was synthesized and cloned into a plasmid vector derived from the pUc plasmid, containing the ampicillin resistance gene and also the Col E1 bacterial origin of replication.

### Transgenesis Vector

The transgenesis vector used for the cloning is derived from the pPolyIII plasmid, which has an ampicillin resistance gene and also the Col E1 bacterial origin of replication. This transgenesis vector contains an expression cassette comprising: a dimer of the sequence of the 5′HS4 insulator of the chicken beta-globin gene (Genbank U78775) (Recillas-Targa et al., Proc. Natl. Acad. Sci., 10: 6883-6888, 2002) upstream of the 6.3 kbp rabbit WAP promoter (whey acidic protein; Genbank X52564) (Rival-Gervier et al., Transgenic Res., 6: 723-730, 2003), the first intron of the rabbit beta-globin gene (Genbank V00882) containing a transcription enhancer; a second transcription enhancer (SUR 1.2.3) containing the 5′UTR sequence of the SV40 early genes, fused with the R region and the start of the U5 region of HTLV-1 (Attal et al., FEBS Lett., 392, 220-224, 1996); a third transcription enhancer (Igp2), derived from the mu region of the mouse IgG heavy chain (Genbank J00440) (Gillies et al., Cell 33, 717-728, 1983) and the human growth hormone transcription terminator (Genbank M13438). This cassette contains an MluI site and an NheI site which are located between the second and the third transcription enhancer; it is flanked on either side by NotI sites which allow the sequences to be excised from the pPolyIII plasmid.

The insert of DNA encoding factor VII, recovered by MluI/NheI digestion from the intermediate vector, was inserted between the MluI and NheI sites of the expression cassette.

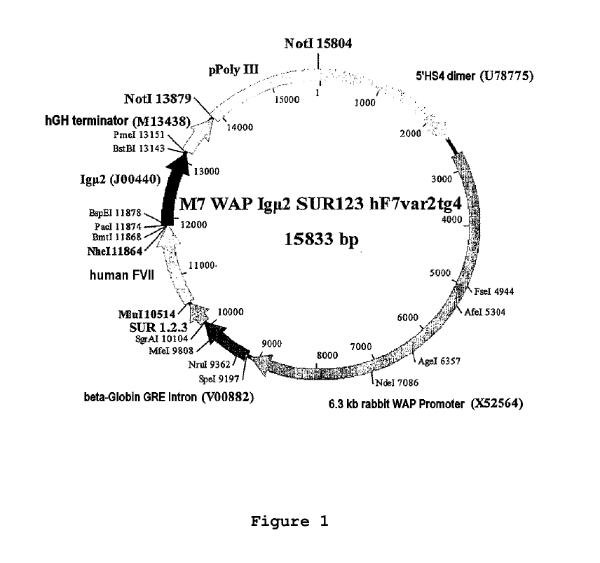

The resulting transgenesis vector is represented in FIG. 1. It contains an insert which is constituted, in its 5′-3′ direction, of i) the dimer of the sequence of the 5′HS4 insulator of the chicken beta-globin gene, ii) the 6.3 kbp rabbit WAP (whey acidic protein) promoter, iii) the intron containing the first transcription enhancer, iv) the second transcription enhancer, v) the human factor VII cDNA, vi) the third transcription enhancer, and vii) the transcription terminator.

As previously, the colonies containing the recombinant vector are selected on the basis of their ampicillin resistance, and then the presence of the insert is verified by restriction fragment analysis and then by sequencing.

### EXAMPLE 2

### Production of Transgenic Rabbits Expressing Human Factor VII in their Mammary Glands

The transgenic rabbits were obtained by the conventional microinjection technique (Brinster et al., Proc. Natl. Acad. Sci., 82: 4438-4442, 1985).

### Preparation of the Inserts for Transgenesis

The transgenesis vector containing the sequence encoding the recombinant factor VII was digested with the NotI restriction enzyme and the insert containing the transgene was isolated on an agarose gel and then purified on ElutipD (Schleicher-Schuell, Ecquevilly, France) in accordance with the manufacturer's instructions, precipitated with ethanol, and then taken up in a 10 mM Tris-HCl buffer containing 0.1 mM EDTA, pH 7.4.

### Preparation of Female Donor and Recipient Rabbits

Embryo-donor female New Zealand rabbits, 16-30 weeks old, are treated subcutaneously for 3 days with porcine FSH (follicle-stimulating hormone) in order to stimulate follicle development. On the third day, the female rabbits receive an intravenous injection of hCG hormone (human chorionic gonadotrophin hormone), and then the females are mated.

The recipient female rabbits are 18-20 weeks old. Synchronized pseudogestation is induced either by keeping the females in a cycle of long days (16 h of light) for one week before mating them with vasectomized males, or by hormone treatment (FSH/LH superovulation).

### Microinjection of the Oocytes Removed and Implantation

On the 4th day, 18-19 h after mating, the embryos are removed from the donor female rabbits in order to perform the DNA microinjection: the DNA microinjection takes place just after the removal of the embryos (15-25 h after mating). The embryos at the one-cell stage are placed in a microdrop of medium under an inverted microscope equipped with Normarsky objectives and micromanipulators. Individual embryos are positioned and secured using a pipette. The transgene, diluted to a concentration that can range from 1 ng/μl to 6 ng/μl, in 10 mM Tris-HCl buffer containing 0.1 mM EDTA, pH 7.4, is microinjected preferentially into the male pronucleus of the embryo using an injection pipette.

Following the microinjection, the embryos are maintained in culture in vitro for 1 to 5 h (35 to 40° C., 3 to 7% CO₂ in air). The quality of the microinjected embryos is then rapidly evaluated under a stereomicroscope. The intact single-cell embryos are then reimplanted under general anesthesia in the lumen of the oviducts of the synchronized recipient female rabbits (10 embryos in each oviduct), using a surgical procedure (the oviducts are exteriorized by laparotomy). Parturition can occur naturally 29-31 days after embryo transfer. If necessary, it can be triggered by injection of oxytocin on the 31st day. The number of young rabbits born relative to the number of embryos reimplanted is about 5% to 20%.

### EXAMPLE 3

### Selection and Characterization of the Transgenic Rabbits

During the embryogenesis process, the microinjected recombinant DNA randomly integrates the genome. The newborn rabbits (10 days) are tested for the presence of the transgene by means of an ear biopsy. The genomic DNA is extracted and a PCR (polymer chain reaction) analysis is carried out using primers specific for the recombinant insert.

The rabbits for which the transgene has been demonstrated are called “F0 Founders”.

The F0-founder lines were also characterized by i) analysis of the number of copies of transgene integrated in their genome, and ii) determination of the number of integration sites.

The number of copies of the transgene integrated into the genome of each F0-founder line was determined by quantitative PCR and by Southern blotting. This number varies, according to the line, from 1 copy per cell to more than 200 copies per cell.

The number of integration sites was also determined by Southern blotting. This number varies, according to the line, from 1 to more than 5 sites per genome.

### EXAMPLE 4

### Evaluation of the Expression of Recombinant Human Factor VII in the Milk of the Transgenic Rabbits

Sexually mature F0 rabbits (4 months for the females, 5 months for the males) were crossed with nontransgenic animals by artificial insemination in order to obtain hemizygous F1 descendants, and the level of expression of human factor VII in the milk was evaluated for various F0-founder lines and F1-descendant lines.

To do this, a sample of milk originating from each of the F0 and F1 females selected was centrifuged at 5000×g for 20 minutes at 4° C. in order to separate the milk compounds from the fats. A sample of skimmed milk was diluted in a Laemmli buffer, brought to boiling for 5 minutes, and then separated by SDS-PAGE gel electrophoresis. The proteins were then transferred onto PVDF membranes, and then a Western blotting analysis was carried out using an anti-human factor VII antibody. The detection was carried out by chemi-luminescence (Amersham Biosciences) in order to determine the human factor VII concentration for each of the milks obtained. A factor VII expression level ranging between 90 μg/ml and 11 mg/ml was observed.

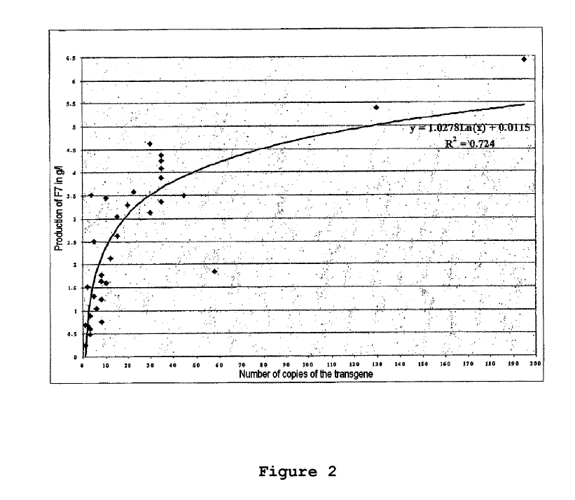

The influence of the number of copies of the transgene on the expression level was also investigated. The results are illustrated in FIG. 2, and show a correlation between the number of copies of transgene and the factor VII expression level in the milk of the transgenic animals.

1. A transgenic rabbit, characterized in that it contains, in its genome, one or more copies of a transgene comprising a polynucleotide encoding human factor VII, placed under the transcriptional control of a promoter which allows its specific expression in the cells of the mammary glands of said rabbit.

2. A milk of a transgenic rabbit as claimed in claim 1.

3. The use of transgenic rabbit milk as claimed in claim 2, as a starting material for the production of recombinant human factor VII.
